(19) United States
(12) Patent Application Publication
(10) Pub. No.: US 2003/0134269 A1
(43) Pub. Date: Jul. 17, 2003

(54) Cell proliferation ability evaluation method and apparatus

(76) Inventors: Hiroyuki Hirai, Handa-shi (JP); Ryota Umegaki, Toyonaka-shi (JP); Masahiro Kinooka, Toyonaka-shi (JP); Masahito Taya, Toyonaka-shi (JP)

Correspondence Address: Y Rocky Tsao, Fish & Richardson, 225 Franklin Street, Boston, MA, 02110-2804, US

(21) Appl. No.: 10/312,409

(22) PCT Filed: Jul. 6, 2001
(86) PCT No.: PCT/JP01/05874

(30) Foreign Application Priority Data
Nov. 22, 2000 (JP) 2000-356344

(51) Int. Cl. C12Q 1/00; G06F 19/00; G01N 33/48; G01N 33/50

(52) U.S. Cl. 435/4; 702/19

(57) ABSTRACT

A method for evaluating the proliferation ability of an entire cell population by observing each anchorage-dependent cell without invading and destroying the cell. The evaluation method includes monolayer-culturing anchorage-dependent cells in a culturing chamber, imaging each cell, calculating an index related to the proliferation ability of each cell using the image of each cell, and evaluating the proliferation ability of the cell population using the index. The index includes an expansion speed (rs) indicative of a change in a projected area of each cell (Sa) during a cell adhesion phase, the number of cells contacting each cell in a cell proliferation phase, and a projected area (Sa′) of each cell in the cell-proliferation phase.

### FIELD OF THE INVENTION

The present invention relates to a method and an apparatus for evaluating the proliferation ability of cells, and more particularly, to a method and an apparatus for evaluating the proliferation ability of anchorage-dependent cells when anchorage-dependent cells are monolayer-cultured in a culturing chamber.

### BACKGROUND ART

In a first prior art cell proliferation ability evaluation method, the proliferation ability of cells is evaluated based on a proliferation curve of the number of cells. More specifically, the number of living anchorage-dependent cells in a culturing chamber is continuously monitored to prepare a standard proliferation curve of the cell from the monitoring results. The proliferation ability of the cells is estimated and evaluated by referring to the standard proliferation curve. The number of living cells is calculated by capturing and processing images of the cells, which are adhered to a bottom surface of the culturing chamber. Alternatively, the number of living cells is calculated by counting of the cells, which detached the adhered cells with an release agent, such as trypsin, with a hematocytometer or a Coulter counter. The standard proliferation curve of the cells is prepared by recording the number of living cells once every predetermined period.

In a second prior art evaluation method, the proliferation curve is prepared by examining the acquisition of a labeled cell proliferation marker, such as ³H-thymidine, into the cells.

In a third prior art evaluation method, pigments are used in cells to color the population of the cells, and analysis results (the number of living cells), such as that of a flow cytometer, are used to conduct research on cell division and cell cycle. The proliferation ability of the observed cells is estimated and evaluated from the analysis results.

However, the first prior art evaluation method evaluates the proliferation ability of cells in a culturing chamber only from a simple statistic analysis, which is based on the number of living cells. In other words, the transition of the number of living cells after counting the number of living cells does not reflect the states of the cells in real time, and the proliferation ability of the cells is predicted based on the standard proliferation curve, which is prepared beforehand. As a result, it is difficult to accurately predict a phenomenon that is actually about to occur with the first prior art evaluation method.

As for the second and third prior art evaluation methods, markers and pigments are employed to indirectly quantify the state of the cells. Thus, such evaluation results partially reflect the state of the cells. However, there is a problem in that markers or pigments directly or indirectly damage and destroy the cells.

### SUMMARY OF THE INVENTION

It is an objective of the present invention to provide an evaluation method for accurately evaluating the proliferation ability of a population of anchorage-dependent cells without invading and destroying the cells.

To achieve the above objective, a first embodiment of the present invention provides a method for evaluating the proliferation ability of a population of anchorage-dependent cells monolayer-cultured in a culturing chamber. The method includes culturing the cells in the culturing chamber, imaging each of the cells, calculating an index related to the cell proliferation ability of each of the cells using the image of each cell, and evaluating the proliferation ability of the cell population using the index.

The anchorage-dependent cells include a cell adhesion phase, in which the cells are adhered to a bottom surface of the culturing chamber after inoculation, expand on the bottom surface and stop expanding at a certain point. It is preferred that the calculating step includes the steps of measuring a projected area of each cell on the bottom surface of the culturing chamber during the cell adhesion phase and calculating an expansion speed of each of the cells from change in the projected area, and the index includes the expansion speed.

It is preferred that the index includes the number of contact cells contacting each of the cells during the cell proliferation phase after a first cell division.

It is preferred that the calculating step includes the step of calculating the projected area of each of the cells on the bottom surface of the culturing chamber during the cell proliferation phase after the first cell division and the index includes the projected area during the cell proliferation phase.

It is preferred that the imaging step includes the step of imaging a culturing state including the cells using a CCD camera and the calculating step includes the step of performing image processing on an image of the culturing state to extract an image of each of the cells.

A second embodiment of the present invention provides a method for evaluating the proliferation ability of a population of anchorage-dependent cells. The method includes the steps of inoculating the cells in a culturing chamber, imaging a culturing state in the culturing chamber, extracting an image of each of the cells from the image of the culturing state, calculating an index related to the proliferation ability of each of the cells from the image of each of the cells, and evaluating the proliferation ability of the cell population using the index.

It is preferred that the calculating step includes the step of calculating a projected area of each of the cells on a bottom surface of the culturing chamber and the index includes the projected area.

It is preferred that the projected area includes a projected area of each of the cells during a cell proliferation phase after a first cell division.

It is preferred that the calculating step includes the step of calculating the number of cells contacting each of the cells and the index includes the number of the contact cells.

The cells include a cell adhesion phase, in which the cells are adhered to the bottom surface of the culturing chamber after inoculation, expand on the bottom surface, and stop expanding at a certain point. It is preferred that the number of the contact cells includes the number of cells contacting each of the cells during the cell adhesion phase.

It is preferred that the imaging step includes imaging the culturing state every predetermined period.

It is preferred that the calculating step includes the step of calculating the projected area of each of the cells on the bottom surface of the culturing chamber every predetermined period and the evaluating step includes the step of evaluating the proliferation ability of the cell population based on a change in the projected area.

It is preferred that the change in the projected area includes changing speed of the projected area of each of the cells during the cell adhesion phase.

It is preferred that the calculating step includes the step of calculating the number of contact cells every predetermined period and the evaluating step includes the step of performing evaluation based on a change in the number of contact cells.

A third embodiment of the present invention provides an apparatus for evaluating the proliferation ability of a population of anchorage-dependent cells. The apparatus includes an incubator for accommodating a culturing chamber in which the cells are inoculated, an imaging device for imaging a culturing state in the culturing chamber, and a computer connected to the imaging device. The computer analyzes an image of the culturing state and extracts an image of each of the cells, calculates an index related to the proliferation ability of the cells from the image of each of the cells, and evaluates the proliferation ability of the cell population using the index.

It is preferred that the calculation circuit calculates a projected area of each of the cells on a bottom surface of the culturing chamber and the index includes the projected area of each of the cells.

It is preferred that the index includes a projected area of the each cell during a cell proliferation phase.

It is preferred that the calculation circuit calculates the number of cells contacting each of the cells and the index includes the number of the contact cells.

It is preferred that the index includes the number of cells contacting each of the cells during a cell adhesion phase.

It is preferred that the imaging device images the culturing state every predetermined period.

It is preferred that the calculation circuit calculates the projected area of each of the cells on the bottom surface of the culturing chamber every predetermined period, and the evaluation circuit evaluates the proliferation ability of the cell population based on a change in the projected area.

It is preferred that the index includes changing speed of the projected area of each of the cells during the cell adhesion phase.

It is preferred that the calculation circuit calculates the number of the contact cells every predetermined period, and the evaluation circuit performs evaluations based on a change in the number of the contact cells.

It is preferred that the imaging device is arranged under the culturing chamber and is a CCD camera for imaging the cells adhered to the bottom surface of the culturing chamber.

It is preferred that the computer further includes a display for displaying an evaluation result.

A fourth embodiment of the present invention provides a computer-readable recording medium storing a program for evaluating the proliferation ability of a population of anchorage-dependent cells. The program causes a computer to execute the steps of analyzing an image of a culturing state in the culturing chamber to extract an image of each of the cells, calculating an index related to the proliferation ability of the cells from the image of each of the cells, and evaluating the proliferation ability of the cell population using the index.

### BRIEF DESCRIPTION OF THE DRAWINGS

FIG. 1a is a graph schematically showing the transition of a projected area of each cell in one embodiment of the present invention.

FIG. 1b is a graph showing the results of test 1 in an example.

FIGS. 2a and 2b are cross-sectional views schematically showing cells in a culturing chamber of the embodiment.

FIGS. 2c to 2f are enlarged cross-sectional views of the cells in the culturing chamber.

FIG. 3 is a semilogarithmic graph showing the results of test 2 in an example.

FIG. 4 is a graph showing the results of test 3 in an example.

FIG. 5 is a graph showing the results of the test 3 in an example.

FIG. 6 is a semilogarithmic graph showing the results of test 4 in an example.

FIG. 7 is a graph showing the results of test 5 in an example.

FIG. 8 is a graph showing the results of test 6 in an example.

FIG. 9 is a graph showing the results of the test 6 in an example.

FIG. 10 is a schematic diagram of an evaluation apparatus in one embodiment of the present invention.

FIG. 11 is a flow chart of an evaluation method in one embodiment of the present invention.

### BEST MODE FOR CARRYING OUT THE INVENTION

One embodiment according to the present invention will now be described.

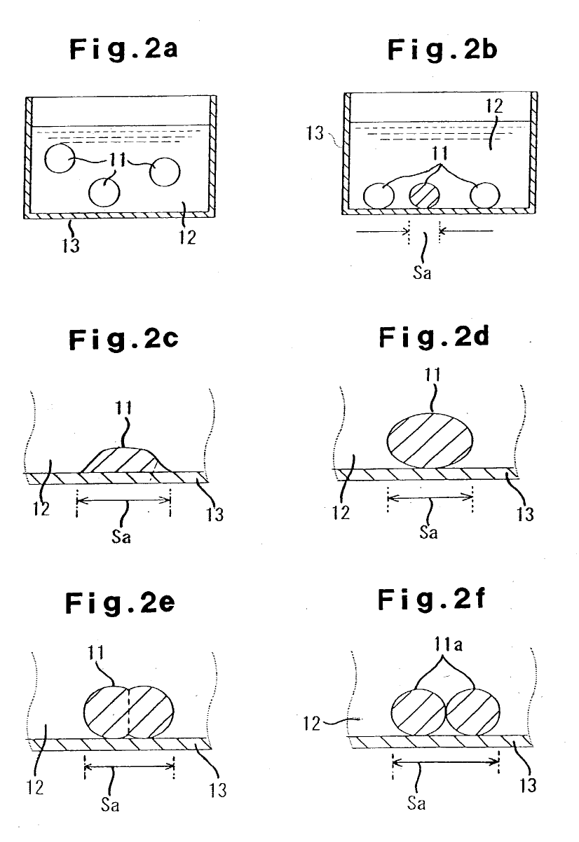

In the cell proliferation ability evaluation method of the embodiment, the proliferation ability of an entire cell population is evaluated when anchorage-dependent cells are monolayer-cultured. In the evaluation method, each cell, which is cultured in a culturing chamber 13, is observed, as shown in FIGS. 2a and 2b. The observation result of each cell is used to evaluate the proliferation ability of the entire cell population in the culturing chamber 13 in a quantified manner.

As shown in FIGS. 2a and 2b, anchorage-dependent cells (hereinafter, described as cells) 11 are monolayer-cultured in the culturing chamber 13, which is filled with culture medium 12. The cells 11 are, directly or by means of a extracellular matrix, adhered to a bottom surface of the culturing chamber 13, and the cells 11 are cultured on the bottom surface of the culturing chamber 13.

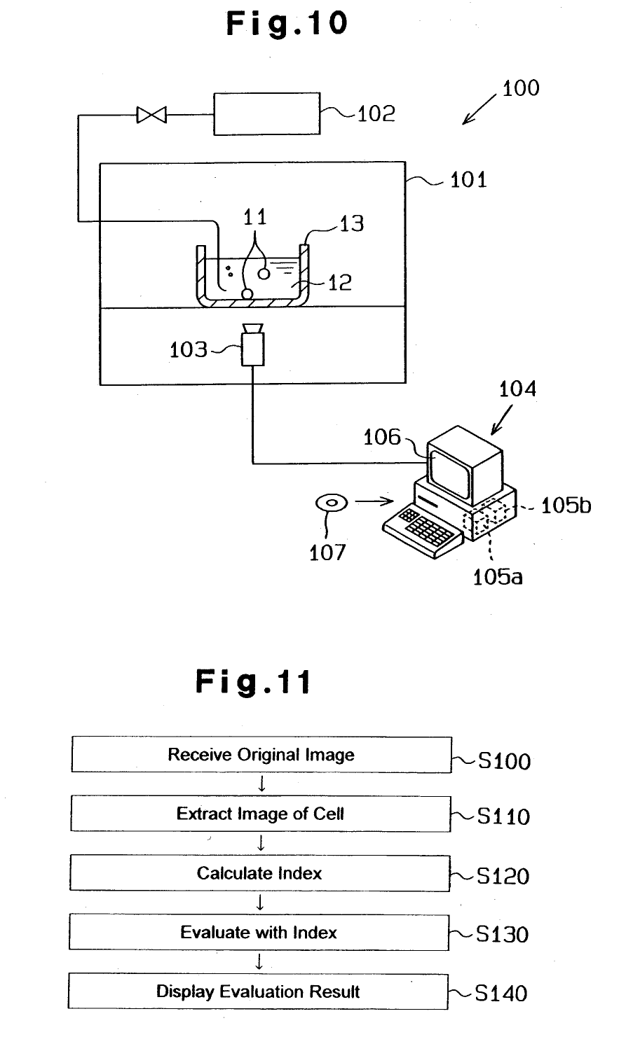

The proliferation ability of the cells is evaluated by an evaluation apparatus 100 of FIG. 10. The evaluation apparatus 100 includes an incubator 101, which accommodates the culturing chamber 13, a gas supply device 102, which supplies the culturing chamber 13 with gas having a predetermined composition, an imaging device 103, which films the culture state of the cells, and a personal computer 104, which is connected to the imaging device 103.

The incubator 101 provides an environment that is optimal for the proliferation of the cells. The imaging device 103 is located below the culturing chamber 13 and provides the personal computer 104 with an original image including the image of the cells, which are adhered to the bottom of the culturing chamber 13. It is preferred that the imaging device 103 be a CCD camera. It is preferred that the personal computer 104 be an image-processing device, which includes a CPU, a main storage unit 105a, an auxiliary memory 105b, and a display 106.

As shown in FIG. 11, the computer 104 receives the original image from the imaging device 103 (S100), analyzes the original image, and extracts the image of each cell (S110). The computer 104 calculates an index related to the proliferation ability of each cell from the extracted image of the cell (S120). The computer 104 evaluates the proliferation ability of the cell population based on the index (S130). The computer 104 displays the evaluation result on the display 105 (S140).

The computer 104 executes a computer program in which the processing method of FIG. 11 is written. The computer program is stored in and provided from a portable recording medium 107, such as a floppy disk or a CD-ROM, or a main memory or auxiliary memory of a further network-connected computer.

The computer program is copied or installed to the auxiliary memory 105b from the portable recording medium 107 and then loaded to the main memory 105a. Alternatively, the computer program is directly loaded from the portable recording medium 107 to the main memory 105a and then executed.

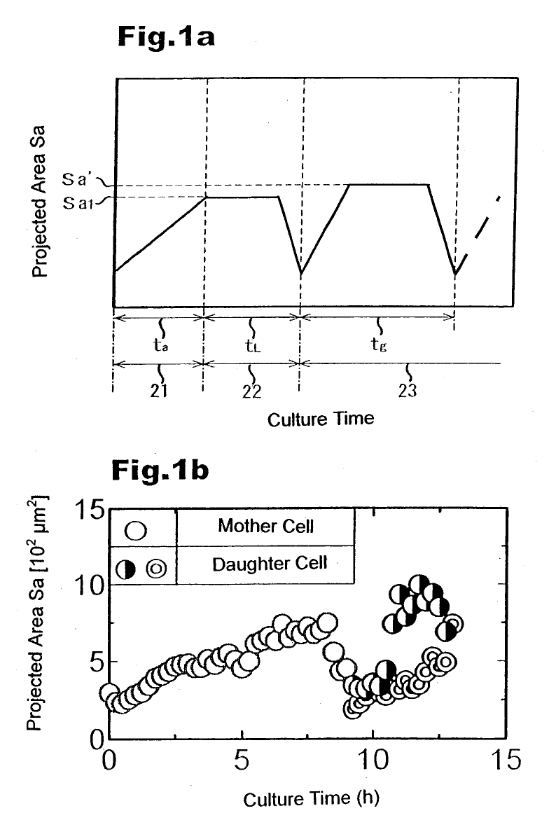

Referring to FIG. 1a, a monolayer-culture process of the cells 11 will now be described. The monolayer-culture process is divided into three stages, a cell adhesion phase 21, a cell lag phase 22, and a cell proliferation phase 23.

Period tₐ represents the cell adhesion phase 21. The initial point of the cell adhesion phase 21 is when the cells 11, which are inoculated into the culturing chamber 13 with the culture medium 12, contacts (adheres to) the bottom surface of the chamber 13. The terminal point of the cell adhesion phase 21 is when planar cell expansion on the bottom surface of the chamber 13 is completed.

The cell adhesion phase 21 will now be described in detail. At the initial stage of the cell adhesion phase 21, or immediately after the cells 11 are inoculated, the cells 11 are substantially spherical and are suspended in the culture medium 12 (see FIG. 2a). The cells 11 then contact and adhere to the bottom surface of the culturing chamber 13 (see FIG. 2b). The cells 11, which are adhered to the bottom surface of the culturing chamber 13, are gradually deformed into a flat shape, as shown in FIG. 2c. This enlarges a projected area Sa of the cells 11. The cell adhesion phase 21 ends when the enlargement of the projected area Sa at the bottom surface of the culturing chamber 13 stops.

The behavior of the cells 11 in this stage slightly differ from that in the normal cell division depending on the extent of the damage, inflicted to the cells 11 when preparing cell suspension for inoculation. The damages are caused by, for example, an isolation operation for isolating the cells 11 from obtained tissue, an enzyme treatment (e.g., protease) for detaching the cells 11 from the bottom surface of the culturing chamber 13, and temperature changes, for returning the refrigerated cells 11 to the original culture temperature. Some of the cells 11 may not live. The cells 11 are adhered to the bottom surface of the culturing chamber 13 after a time substantially proportional to the extent of the damage elapses and live. In contrast, the cells 11 that do not live do not become adhered the bottom surface of the culturing chamber 13 and die.

The cell lag phase 22 is the period from when the cell adhesion phase 21 ends to when a cell division is completed for the first time. In this stage, to adapt to a new environment after being adhered to the bottom surface of the culturing chamber 13, the cell 11 is flat while adhered to the bottom surface of the culturing chamber 13, as shown in FIG. 2c. Accordingly, in this stage, the projected area Sa hardly expands and then proceeds to the next stage while maintaining a state in which the projected area Sa is equal to Sa1, as shown in FIG. 1a. The cell 11 becomes substantially spherical with a short period of time just before cell division, as shown in FIGS. 2d and 2e. Therefore, the projected area Sa temporarily decreases. The cell 11 (mother cell) in the cell lag phase 22 normally completes cell division after a predetermined lag time t₁ for a first time, and is divided into two daughter cells 11a (FIG. 2f).

The cell proliferation phase 23 is the period after the cell lag phase 22 is completed (after the completion of the first cell division). In this stage, the daughter cells 11a repeat cell division every constant generation time tg, unless contacting cells, such as other daughter cells, are adjacent. The generation time tg increase as the number of contact cells increase. The cell division stops when the bottom surface of the culturing chamber 13 becomes confluent, that is, when the entire bottom of the culturing chamber 13 is covered by the monolayer cells 11 and the daughter cells 11a are entirely surrounded by other contact cells.

In the cell proliferation phase 23, the projected area Sa of each of the cells 11 suddenly expands immediately after the cell divisions, as shown in FIG. 1a. A cycle in which the projected area Sa is maintained at a substantially constant value (Sa′) during a predetermined period and is then suddenly decreased just before the next cell division is repeated.

It is preferred that each of the cells 11 in the culturing chamber 13 is continuously imaged and monitored using the CCD camera. Since the generated original image includes images other than those of the cells 11, a proper image processing is performed to specify the projected image of the cells 11, which are adhered to the bottom surface of the culturing chamber 13, and the peripheral outline of the projected image. The cell image is extracted by the image processing of the original image. It is preferred that the projected area Sa of each of the cells 11, which are monitored, be calculated based on the number of pixel elements and the outline of the extracted cell images. It is preferred that the number of contact cells adjacent to each cell 11 be counted from the extracted cell image. Calculation results, or the projected area Sa and/or the number of contact cells, are used as an index for evaluating the proliferation ability of the cells.

As shown in FIG. 1a, the state of the cells 11, including cell divisions, is easily recognized from changes in the projected area Sa when monitoring the projected area Sa of each cell 11. More specifically, the cells 11 are substantially spherical in the most initial stage of the cell adhesion phase 21, and the projected area Sa is thus extremely small. As time elapses, the cells 11 gradually become flat on the bottom surface of the culturing chamber 13. This increases the projected area Sa. When the adhesion phase 21 ends, the cells 11 stop deforming, and the projected area Sa stops expanding.

In the cell lag phase 22, the projected area of the cells 11 remain substantially constant (Sa1). During this period, the cells 11 prepare for the first cell division after they are adhered (specifically, from a DNA synthesis preparation period (G1 period) to a DNA synthesis period (S period) in a cell cycle). When the cells 11 have completely prepared for the cell division, the cells 11 are deformed to be spherical again to mitose for a division preparation period (a G2 period). The G2 period corresponds to the period when the projected area Sa suddenly decreases just before the cell lag phase 22 ends.

Subsequently, the projected area Sa temporarily becomes extremely small in the cell division period (M period: boundary between the cell lag phase 22 and the cell proliferation phase 23 in FIG. 1a), but increases again later. This is because a mother cell 11 is divided into a plurality of the daughter cells 11a (normally, two). The daughter cells 11a are anchored to the bottom surface of the culturing vessel 13 and contact one another at their sides while remaining in substantially the same position (see FIG. 2f). This gradually increases the projected area Sa in the same manner as the above mentioned cell adhesion phase 21.

Next, the cells 11 (the daughter cells 11a) change in the same manner as in the cell lag phase 22. In other words, since the cells 11 prepare for the second cell division after they are adhered, the projected area is maintained at the substantially constant value (Sa′). Then, the cells 11 are deformed to be spherical and mitose. During the cell proliferation phase 23, the cells 11 (11a) repeatedly undergo a series of cell division processes to proliferate until becoming confluent.

The proliferation ability of the entire cell population, which is adhered in the culturing chamber 13, largely depends on an expansion speed rs, which represents the change (increase) in the projected area Sa of each of the cells 11 in the cell adhesion phase 21. Accordingly, the expansion speed rs may be used as an index for evaluating the proliferation ability of the entire cell population. The expansion speed rs, which is a value calculated by dividing the difference between the maximum value and minimum value of the projected area Sa in the cell adhesion phase 21 by a culture period tₐ, corresponds to the inclination (an average value) of the graph of FIG. 1a during the cell adhesion phase 21.

In addition, the proliferation ability of the entire cell population largely depends on the number of cells contacting each cell 11 in the cell proliferation phase 23. Therefore, the number of cells contacting each cell 11 may be used as an index for evaluating the proliferation ability of the entire cell population. That is, the anchorage-dependent cells 11 have a characteristic in which they are cultured in monolayer and, a characteristic in which a plurality of the daughter cells 11a live contacting each other after cell division. When there is no further surplus space that allows the daughter cells 11a to be adhered, the contact inhibition prevents the daughter cells 11a from undergoing cell division. The contact inhibition escalates in proportion to the increase in the number of cells contacting each cell 11.

Further, the proliferation ability of the entire cell population largely depends on the projected area Sa′ of each cell 11 in the cell proliferation phase 23. Therefore, the projected area Sa′ in the cell proliferation phase 23 may be used as an index for evaluating the proliferation ability of the entire cell population taking into consideration the cell life. In other words, the cells 11 have a characteristic in which they culture in monolayer and a characteristic in which they have a finite cell life, which is directly related to the number of cell divisions after cell differentiation. When the cells 11 reach the limit of the cell life, they loose the proliferation ability. The length of the cell life is substantially irrelevant to cell strains and is negatively proportional to the projected area Sa′ of each cell 11.

The projected area Sa′ represents the projected area of each cell in the cell proliferation phase (G0 period, G1 period, and S period) 23, which is substantially constant. The projected area Sa′ in the cell proliferation phase 23 normally tends to increase little by little as the number of cell divisions increases. It is, therefore, preferred that the average value of a plurality of the projected areas Sa′ subsequent to the first cell division be used to evaluate the proliferation ability. However, in the period between the inoculation and the state of confluent, the difference between the previous value and the current value of the projected area Sa′ (the difference of increase) is relatively small. Accordingly, it may be possible to evaluate the proliferation ability using the projected area Sa′ just before the second cell division. By using the projected area Sa′ just before the second cell division, evaluation of the proliferation ability of the cell population at an early stage of culturing is facilitated.

During the proliferation ability evaluation, the same type of cells 11 are tentatively used and the proliferation of each cell is monitored under the same culturing condition to prepare the proliferation curve of the entire cell population (preliminary experiment result). From the preliminary experiment results, an index indicative of the proliferation ability of the entire cell population, such as the generation time tg, the ratio of cell division Nₜ/Nₒ until a predetermined time t, a superficial doubling time td, an average specific growth rate μ, the proliferation rate Y (t), or the average number of cell divisions Nd may be obtained. The actual proliferation ability of the cells 11 is evaluated in comparison with the results of the preliminary experiment.

Further, to reduce evaluation errors, it is preferred that an average of observation results for the cells 11, which are selected at random, be obtained. In addition, it is preferred that the proliferation ability be evaluated using a combination of the expansion speed rs, the number of contact cells, and the projected area Sa′ since the evaluation becomes further accurate.

The superficial doubling time td is calculated as follows. First, a cell 11 is inoculated into the culturing chamber 13 at cell inoculation concentration Xo (cells/cm²), in which the cell 11 does not contact adjacent cells 11. The average specific growth rate μ is measured by monitoring change in the cell adhesion concentration Xa (cells/cm²), when the cells undergo logarithmic proliferation. The superficial doubling time td is calculated based on the average specific growth rate μ.

The proliferation rate Y (t) is calculated as follows. First, a cell 11 is inoculated at a certain cell inoculation concentration Xo and is cultured. The cell adhesion concentration Xₐt in a predetermined culture time t during the cell proliferation phase 23 is measured. The proportion of the cell inoculation concentration Xo relative to the cell adhesion concentration Xₐt, that is, the adhesion ratio (Xₐt/Xo), is calculated. The proliferation rate Y (t) is determined based on the adhesion rate (Xₐt/Xo).

The average number of cell divisions Nd is calculated by substituting Xₐ and Xo to following equation (2). This is based on the fact that the cell adhesion concentration Xa and the cell inoculation concentration Xo have the relation represented by the following equation (1).

Xₐ=Xₒ×2^{ND} (1)

Nd=ln(Xₐ/Xo)/ln2 (2)

The cell proliferation ability evaluation method may increase data types of the preliminary experiment results to evaluate the metabolic activity of the entire cell population, the cell life, the stress recovering ability, the extent of differentiation, or the quality of the cells when used as the tissue for transplant (the recovering speed of an affected area), in addition to the proliferation ability of cells.

In the above embodiment, the proliferation ability of cells is evaluated based on the projected area. However, an adhesion area of the cells 11 may be used instead of the projected area. In this case, it is preferred that a confocal scanning laser microscope be used instead of the CCD camera. The confocal scanning laser microscope clearly images a surface, to which the cells are adhered, and the profile about the adhesion surface.

The above embodiment has the following advantages.

In the embodiment of the cell proliferation ability evaluation method, the proliferation ability of the entire cell population in the culturing chamber 13 is evaluated using observation results of the cell images, which are formed by imaging each cell cultured in the culturing chamber 13, in a quantified manner. Thus, the proliferation ability of the entire cell population is easily and accurately evaluated without invading (pigmenting) and destroying each cell 11.

Since the expansion speed rs of each cell 11 is measured in the cell adhesion phase 21, the proliferation ability of the cells 11 is easily determined in an early stage of culturing.

Since the number of cells contacting each cell 11 is counted in the cell proliferation phase 23, the proliferation ability of the cells 11 in a latter stage of culturing is easily measured.

Since the projected area Sa′ of each of the cells 11 is measured in the cell proliferation phase 23, the proliferation ability (cell life) of each cell 11 after the measurement time is easily estimated.

### EXAMPLES

The embodiment will be described in detail using the following examples.

<Test Conditions>

Cell: Mouse NIH 3T3 p-7 cl-3 IFO 50019 cell strain (Institute for Fermentation)

Culture medium: DMEM+10% bovine neonatal serum (Sigma Corp.)

Culturing chamber: 25 cm² T-flask (Falcon Corp.)

Amount of culture medium: about 10 ml (depth of 4 mmm in T-flask)

Culture temperature: 37° C. (humidity 100%)

Ventilation condition: air (5% of Co₂ included), current velocity 5 ml per minute

Cell inoculation concentration X₀: 1.0×10⁴ cells/cm²

Imaging device: CCD camera (Tokyo Electronic Industry Co., Ltd.)

Imaging area: 900 μm×680 μm (6.1×10⁻³ cm²)

Image processing device: personal computer (IBM Corp.)

Image processing 1: quantification of projected area Sa of each cell

original image (captured every 10 minutes) background separation process→look-up table conversion→smoothing process→binary extracting process→isolated point removal process→closing process→padding process→area extracting process→pixel number measurement

Image processing 2: The number of all cell in an imaging area was measured and the cell adhesion concentration Xₐ (cells/cm²) was calculated from the number of all cell.

<Test 1: Observing a Cell in an Initial Culture Stage>

Cells 11 were inoculated in a culturing chamber 13. Then, the imaging device 103 was used to image one of the cells 11, which was adhered to the bottom surface of the chamber 13. The image processing apparatus 104 then conducted the image processing 1 to measure a projected area Sa. Changes in the projected area Sa is shown in FIG. 1b. The culture time began when the cells 11 were adhered to the bottom surface of the culturing chamber 13.

It was confirmed that the cells 11 were spherical and gradually started to expand on the bottom surface of the culturing chamber 13 by viewing the original image of the cells 11. The graph of FIG. 1b shows that the projected area Sa was relatively small during the initial stage of culturing and expanded in a substantially linear manner until about 6 hours elapsed from when the culturing started. When the culture time was between about 6 to 8 hours, the projected area Sa was substantially constant. When the culture time was between about 8 to 9 hours, the projected area Sa suddenly decreased. Accordingly the cell division was confirmed.

The graph of FIG. 1b shows that, with regard to the cells 11, the cell adhesion phase 21 corresponds to the period between about 0 to 6 hours after the cells are adhered and that the cell lag phase 22 correspond to the period between about 6 to 9 hours after the cells are adhered, the cell proliferation phase 23 corresponds to after 9 hours from the adherence of the cells. In addition, for the cell 11, time tₐ of the cell adhesion phase 21 was about 6 hours, a lag time t_{L} was about 3 hours, and an expansion speed rs was about 62 μm²/h.

<Test 2: Preparing Proliferation Curve of a Cell Population>

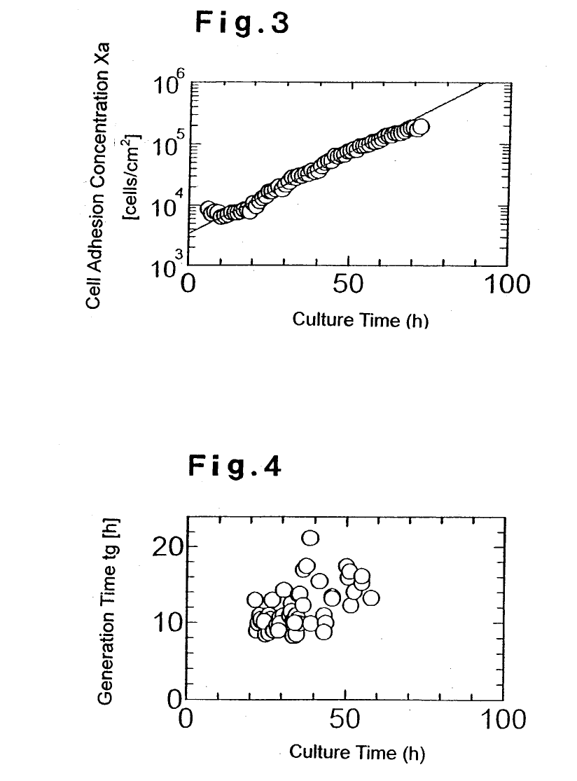

During and after the observation of the test 1, the cell proliferation ability of an entire cell population was evaluated by continuously conducting follow-up research on the cell population, which was inoculated into the culturing chamber 13. More specifically, the entire cell population, which was inoculated into the culturing chamber 13, was observed by a processing method of the image processing 2 to measure the adhesion concentration Xₐ of the cells 11, which were adhered to the bottom surface of the culturing chamber 13 with time. This prepared the proliferation curve of the cell population. The proliferation curve is shown in FIG. 3.

The graph of FIG. 3 shows that the cell population undergoes exponential proliferation during the period between 20 to 60 hours after the cells were inoculated. The inclination of the proliferation curve in this period indicated that the superficial doubling time td was about 11.2 hours. Thus, it is predicted that the cell population with the expansion speed rs of about 62 μm²/h proliferates according to the proliferation curve shown in FIG. 3. Further, the superficial doubling time td is predicted to be about 11.2 hours when the cell population undergoes the exponential proliferation.

<Test 3: Observing Each Cell During Latter Stage of Culturing>

In a latter stage of culturing (20 to 60 hours) after the observation of the test 1, each cell (n=60) in the culturing chamber was observed according to a processing method of the image processing 1 to measure the generation time tg. The relation between the generation time tg and culture time is shown in FIG. 4. The graph of FIG. 4 illustrates that the generation time tg of each cell gradually became longer as the culture time increases.

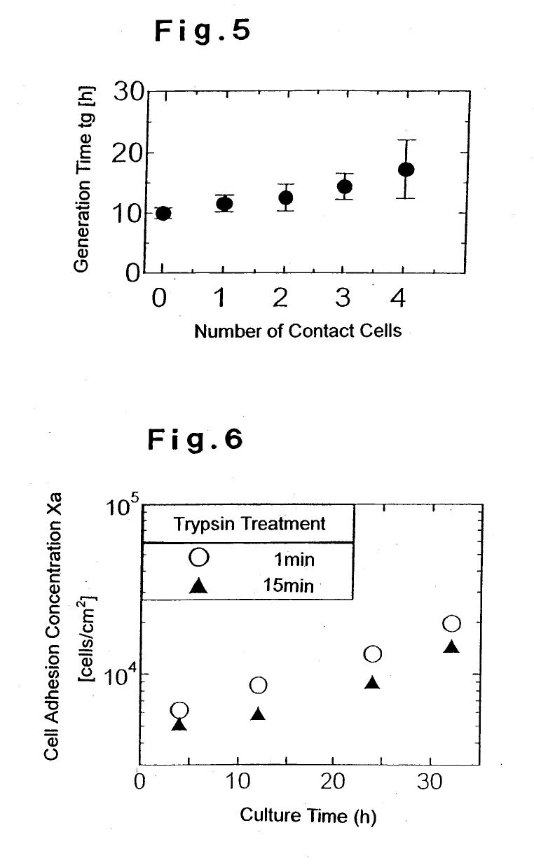

The original image of each cell was viewed to measure the number of contact cells. The relation between the number of contact cells and the generation time tg was illustrated in FIG. 5. The graph of FIG. 5 illustrates that the generation time tg increases as the number of contact cells increases. Therefore, it is estimated that the prolonged generation time tg was mainly caused by the contact inhibition.

<Test 4: Evaluation Test 1 of the Proliferation Ability Using Trypsin>

A trypsin treatment was performed on cells, which were cultured in a culturing chamber for one minute, to detach the cells from a bottom surface of the culturing chamber. The cell suspension for sub culture was prepared from the cells, which were detached. The cell suspension was inoculated into another new culturing chamber. Hereinafter, the cells are referred to as one-minute trypsin treatment cells. In the same manner, the cells, which were sub-cultured after the trypsin treatment for 15 minutes, was prepared. Hereinafter, the cells are referred to as 15-minuetes trypsin treatment cells.

With regard to each cell in the culturing chamber (n=9, respectively), the average value of the expansion speed rs was calculated by monitoring a projected area Sa of each cell using the imaging device 103 and the image processing 1. The average value for the expansion speed rs of one-minute trypsin treatment cells was about 72 μm²/h, and the average value for the expansion speed rs of 15-minutes trypsin treatment cells was about 24 μm²/h.

With regard to the one-minute trypsin treatment cells and 15-minutes trypsin treatment cells, the cell adhesion concentration Xₐ was chronologically monitored by means of a processing method of the image processing 2 when calculating the expansion speed rs. As shown in the graph of FIG. 6, the relation between the cell adhesion concentration Xₐ and culture time was plotted to prepare a proliferation curve of each cell population. The graph in FIG. 6 illustrates that the expansion speed rs of one-minute trypsin treatment cells is higher than that of 15-minutes trypsin treatment cells and that the proliferation ability of the entire cell population increases as the expansion speed rs increases. Accordingly, it is understood that the expansion speed rs of each cell in the initial stage in culturing significantly affects the proliferation ability of the entire cell population.

<Test 5: Evaluation Test II for the Proliferation Ability Using Trypsin>

Cells, which were cultured in the culturing chamber, were detached from the bottom surface of a culturing chamber by properly processing them at different trypsin treatment periods. The detached cells were used to prepare the sub-culturing cell suspension for every trypsin treatment period. Each cell suspension was inoculated into a new culturing chamber. With respect to each cell in the culturing chamber (n=8, respectively), the imaging device 103 and the image processing 1 were used to monitor the projected area Sa to calculate the average value of the expansion speed rs.

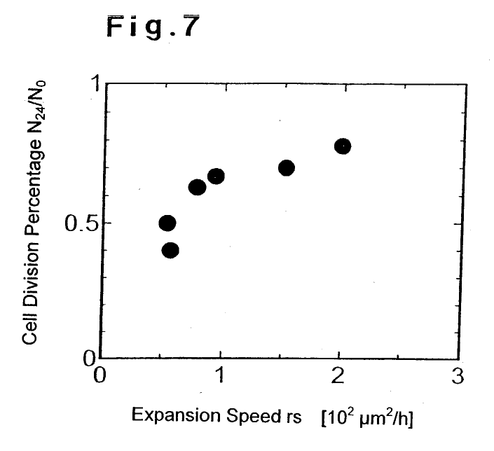

Further, with regard to each cell in the culturing chamber, the number of cell division (0 or 1) was monitored until 24 hours after inoculation. The cell division percentage N₂₄/Nₒ was calculated from the analyzed cell number Nₒ and the number of cell division N₂₄ within 24 hours in each culturing chamber. The relation between the expansion speed rs and the cell division percentage N₂₄/Nₒ is shown in FIG. 7.

FIG. 7 illustrates that the cell division percentage N₂₄/Nₒ increases as the expansion speed rs increases. Accordingly, it is understood that the proliferation ability of the entire cell population may be evaluated from the expansion speed rs of each cell in the initial stage of culturing.

<Test 6: Evaluation Test for the Cell Proliferation Ability in the Series of Sub-Culturing>

<Test Conditions>

Cell: Human keratinocyte ((a) normal human newborn prepuce epidermic ketatinocyte cell strain, (b) normal adult human breast epidermic ketatinocyte adult cell strain) (Kurabo Industries Ltd.)

Culture medium: serum-free medium for keratinocyte (HuMedia-KG2, Kurabo Industries Ltd.)

The other test conditions are the same as the above conditions.

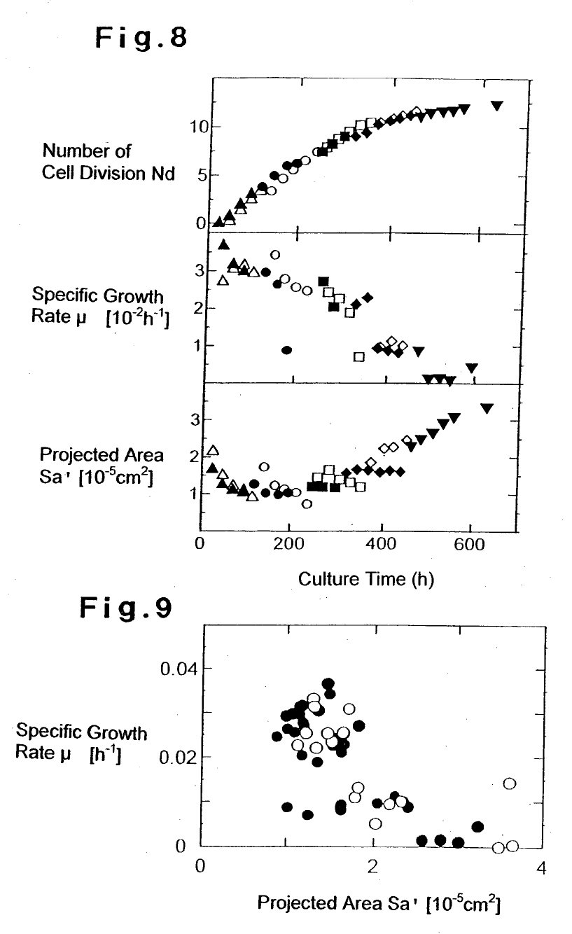

With regard to each cell (n=20), which was cultured in a culturing chamber, the imaging device 103 and the image processing 1 were used to chronologically monitor the projected area of each cell in the cell proliferation phase 12 and calculate the average projected area Sa′. This operation was performed on two kinds of cell strains (a) and (b), the donors of which were of different ages. In addition, the cells were monitored over a long period to examine the average projected area Sa′ by performing a plurality of sub-culturing operations. The results are shown in FIG. 8. The data of the cell strain (a) derived from a newborn is indicated by black symbols including ▴, ●, ▪, ♦, and ▾, while data of the cell strain (b) derived from 20-year-old adult is indicated by white symbols including Δ, ∘, □, ⋄, and ∇. The different symbols were used every time sub-culturing was performed.

FIG. 8 shows that the average projected area Sa′ gradually decreased in the first sub-culturing, but gradually increased after the second sub-culturing. The temporal decrease in the projected area Sa′ of the first sub-culturing is believed to have been caused by the use of the cells in a frozen state. In other words, in the initial stage of the first sub-culturing the cells were relieved from damages (stress) caused by freezing and thawing and recovered their original projected area. It is believed that the recovery caused the temporal decrease in the projected area Sa′.

When calculating the projected area Sa′, the cell adhesion concentration Xₐ was chronologically monitored in accordance with the image processing 2 to calculate the average specific growth rate μ and the average number of cell divisions Nd of the cell population. The results are shown in FIG. 8. According to the result of FIG. 8, the two kinds of cell strains (a) and (b) were similar in the specific growth rate μ and the number of cell divisions Nd. More specifically, as the culture time increases, the average number of cell divisions Nd and the projected area Sa′ of the cells increases. However, the average specific growth rate p of the cell population decreases.

FIG. 9 illustrates the relation between the projected area Sa′ and the specific growth rate μ. In FIG. 9, the data of the cell strain derived from a newborn (a) is indicated by ●, while the data of the cell strain derived from an adult (b) is indicated by ∘.

FIG. 9 shows that the projected area Sa′ of the cells to the average specific growth rate μ of the cell population is negatively proportional and that the relation between the specific growth rate μ and the projected area Sa′ is substantially the same regardless of the type of cell strain. Accordingly, it is understood that the measuring of the projected area Sa′ of the cells at a certain point enables prediction of how the cells would proliferate in the future.

Further, the projected area Sa′, the specific growth rate μ, and the number of cell divisions Nd tend to change in substantially the same manner regardless of differences in cell stains, as shown in FIGS. 8 and 9. This enables easy prediction of the cell life using the projected area Sa′ of the cells, the specific growth rate μ and the number of cell divisions Nd.

The culture cell evaluation method and evaluation apparatus according to the present invention has the following advantages.

Each anchorage-dependent cell is observed without being invaded and destroyed. Thus, the proliferation ability of the entire anchorage-dependent cell population is easily and accurately recognized. The proliferation ability of the anchorage-dependent cells is easily determined in the initial stage during culturing. The proliferation ability of the anchorage-dependent cells in the latter stage of culturing is easily mesured. Further, the proliferation ability of the anchorage-dependent cells in the cell proliferation phase is easily estimated.

The preferred embodiments of the present invention are described in connection with the drawings, but the present invention is not limited to the foregoing, and the attached claims and alternations are permitted.

1. A method for evaluating the proliferation ability of a population of anchorage-dependent cells being monolayer-cultured in a culturing chamber, the method comprising:
culturing the cells in the culturing chamber;
imaging each of the cells;
calculating an index related to the cell proliferation ability of each of the cells using the image of each cell; and
evaluating the proliferation ability of the cell population using the index.

2. The evaluation method according to claim 1, characterized in that the cells include a cell adhesion phase, in which the cells are adhered to a bottom surface of the culturing chamber after inoculation, expand on the bottom surface, and stop expanding at a certain point, wherein the calculating step includes the steps of:
measuring a projected area of each cell on the bottom surface of the culturing chamber during the cell adhesion phase; and
calculating an expansion speed of each of the cells from change in the projected area, wherein the index includes the expansion speed.

3. The evaluation method according to claim 1 or claim 2, characterized in that the index includes the number of contact cells contacting each of the cells during the cell proliferation phase after a first cell division.

4. The evaluation method according to any one of claims 1 to 3, characterized in that the calculating step includes the step of calculating the projected area of each of the cells on the bottom surface of the culturing chamber during the cell proliferation phase after the first cell division, wherein the index includes the projected area during the cell proliferation phase.

5. The evaluation method according to any one of claims 1 to 4, wherein the imaging step includes the step of imaging a culturing state including the cells using a CCD camera, the calculating step includes the step of performing image processing on an image of the culturing state to extract an image of each of the cells.

6. A method for evaluating the proliferation ability of a population of anchorage-dependent cells, the method comprising the steps of:
inoculating the cells in a culturing chamber;
imaging a culturing state in the culturing chamber;
extracting an image of each of the cells from the image of the culturing state;
calculating an index related to the proliferation ability of each of the cells from the image of each of the cells; and
evaluating the proliferation ability of the cell population using the index.

7. The evaluation method according to claim 6, wherein the calculating step includes the step of calculating a projected area of each of the cells on a bottom surface of the culturing chamber, wherein the index includes the projected area.

8. The evaluation method according to claim 7, wherein the projected area includes a projected area of each of the cells during a cell proliferation phase after a first cell division.

9. The evaluation method according to claim 6, wherein the calculating step includes the step of calculating the number of cells contacting each of the cells, wherein the index includes the number of the contacting cells.

10. The evaluation method according to claim 9, wherein the cells include a cell adhesion phase, in which the cells are adhered to the bottom surface of the culturing chamber after inoculation, expand on the bottom surface, and stop expanding at a certain point, wherein the number of the contacting cells includes the number of cells contacting each of the cells during the cell adhesion phase.

11. The evaluation method according to claim 6, wherein the imaging step includes imaging the culturing state every predetermined period.

12. The evaluation method according to claim 11, wherein the calculating step includes the step of calculating the projected area of each of the cells on the bottom surface of the culturing chamber every predetermined period, wherein the evaluating step includes the step of evaluating the proliferation ability of the cell population based on a change in the projected area.

13. The evaluation method according to claim 12, wherein the change in the projected area includes changing speed of the projected area of each of the cells during the cell adhesion phase.

14. The evaluation step according to claim 11, wherein the calculating step includes the step of calculating the number of contact cells every predetermined period, and wherein the evaluating step includes the step of performing evaluation based on a change in the number of contact cells.

15. An apparatus for evaluating the proliferation ability of a population of anchorage-dependent cells, the apparatus being characterized by:
an incubator for accommodating a culturing chamber in which the cells are inoculated;
an imaging device for imaging a culturing state in the culturing chamber; and
a computer connected to the imaging device, wherein the computer;
analyzes an image of the culturing state and extracts an image of each of the cells;
calculates an index related to the proliferation ability of the cells from the image of each of the cells; and
evaluates the proliferation ability of the cell population using the index.

16. The evaluation apparatus according to claim 15, wherein the calculation circuit calculates a projected area of each of the cells on a bottom surface of the culturing chamber, wherein the index includes the projected area of each of the cells.

17. The evaluation apparatus according to claim 16, wherein the index includes a projected area of the each cell during a cell proliferation phase.

18. The evaluation apparatus according to claim 15, wherein the calculation circuit calculates the number of cells contacting each of the cells, and wherein the index includes the number of the contacting cells.

19. The evaluation apparatus according to claim 18, wherein the index includes the number of cells contacting each of the cells during a cell adhesion phase.

20. The evaluation apparatus according to claim 15, wherein the imaging device images the culturing state every predetermined period.

21. The evaluation apparatus according to claim 20, wherein the calculation circuit calculates the projected area of each of the cells on the bottom surface of the culturing chamber every predetermined period, and wherein the evaluation circuit evaluates the proliferation ability of the cell population based on a change in the projected area.

22. The evaluation apparatus according to claim 21, wherein the index includes changing speed of the projected area of each of the cells during the cell adhesion phase.

23. The evaluation apparatus according to claim 20, wherein the calculation circuit calculates the number of the contacting cells every predetermined period, and wherein the evaluation circuit performs evaluations based on a change in the number of the contacting cells.

24. The evaluation apparatus according to claim 15, wherein the imaging device is arranged under the culturing chamber and is a CCD camera for imaging the cells adhered to the bottom surface of the culturing chamber.

25. The evaluation apparatus according to claim 15, wherein the computer further includes a display for displaying an evaluation result.

26. A computer-readable recording medium storing a program for evaluating the proliferation ability of a population of anchorage-dependent cells, the program causes a computer to execute the steps of:
analyzing an image of a culturing state in the culturing chamber to extract an image of each of the cells;
calculating an index related to the proliferation ability of the cells from the image of each of the cells; and
evaluating the proliferation ability of the cell population using the index.
